(19) United States
(12) Patent Application Publication
(10) Pub. No.: US 2022/0270253 A1
(43) Pub. Date: Aug. 25, 2022

(54) METHOD FOR PRODUCING A COMPUTER MODEL FOR AN ABUTMENT AND FOR PRODUCING AN ABUTMENT

(71) Applicant: EAP® PRODUKTIONS- UND PATENTVERWERTUNGS- GMBH, HALL IN TIROL (AT)

(72) Inventor: Mario KERN, Kramsach (AT)

(21) Appl. No.: 17/684,953

(22) Filed: Mar. 2, 2022

Related U.S. Application Data
(63) Continuation of application No. PCT/AT2020/060328, filed on Sep. 2, 2020.

(30) Foreign Application Priority Data
Sep. 3, 2019 (AT) A 50767/2019

(51) Int. Cl. G06T 7/00; A61C 8/00; G06T 17/00

(52) U.S. Cl. CPC G06T 7/0012; A61C 8/0068; A61C 8/0022; G06T 17/00; A61C 2008/0084; G06T 2210/41

(57) ABSTRACT

A method produces a three-dimensional computer model of an abutment for a tooth implant by a computer, and the abutment has a base, a connecting structure, a platform surface of coronal appearance, and a screw channel. The method includes: providing a three-dimensional representation of a jaw of the patient to be read by the computer, choosing a definition of a preparation margin depending upon the patient's individual gum line, defining a three-dimensional geometry of the abutment above the preparation margin by an operator of the computer or by an algorithm, defining an emergence profile of the abutment by an operator of the computer or by an algorithm, and providing the three-dimensional computer model by the computer. A plate-like design of the platform surface is provided below the preparation margin for the three-dimensional geometry of the platform.

### BACKGROUND OF THE INVENTION

The invention concerns a method of producing an abutment as well as a computer program product, a computer-readable storage medium, a computer-readable data carrier and a data carrier signal which transmits such a computer program product and a computer configured for carrying out one of the methods and/or for executing such a computer program product.

The term abutment (which can sometimes also be referred to as the “attachment”) is used in dentistry to denote the connecting element between a pin-shaped dental implant and a prosthetic fitting (for example a single tooth fitting, a bridge fitting, dentures and so forth). The connection to the prosthetic fitting can be effected directly or by way of a mesostructure.

An abutment can be found in EP 2 825 124 B1. The abutment disclosed in that document has a platform with a plate-like configuration of the coronally looking platform surface which promotes the growth of cells and allows support for the mesostructure or a prosthetic fitment on the coronally looking platform surface. The possibility of subsequent treatment of the abutment affords individualizability of the abutment for a given patient.

The term emergence profile is used in dental implant treatment to denote the three-dimensional configuration of the gingiva where the abutment is to be placed, in a coronally extending region from the dental implant to the gingiva line.

Besides prefabricated standard abutments which have to be manually retreated in the dental laboratory in a complicated and expensive procedure in order to adapt them to the specific factors at the treatment location in the jaw of the patient it is already known to produce directly patient-individualized abutments. That is effected by way of a three-dimensional computer module, readable by a computer, of the patient-individualized abutment to be produced. The three-dimensional computer model is either produced on the basis of an analog dental imprint of the jaw of the patient, which is digitized, or it is provided directly in digital form (for example using an intraoral scanner or laboratory scanner).

The gingiva configuration which is individual for the patient is defined by an operator (for example a dental technician or a dentist) or by an algorithm, where the abutment is to be placed.

In addition the three-dimensional geometry of the abutment above a preparation limit which is predetermined by the individual gingiva configuration is to be defined by the operator or an algorithm. Definition of the three-dimensional geometry of the abutment can include:
Defining a position of a screw passage extending in the abutment for fixing the abutment to a pin-shaped dental implant. Usually, the position is defined relative to an orientation of the dental implant.
Defining a width of a coronally looking platform surface of the abutment.
Defining a height of the abutment.
Defining a width of a central raised portion of the abutment, that has the screw passage.

In addition the emergence profile of the abutment is to be defined by an operator of the computer or by an algorithm.

The emergence profile, i.e. the external geometry of the abutment beneath the preparation limit as far as the pin-shaped dental implant, was previously predetermined with a standard form and the region between the emergence profile and the screw passage was defined as a filled body so that the operator of the computer had no room for maneuver here at all. As a result a preparation limit which in itself extends too deeply was frequently adopted in practice in order to enlarge that region which could be selected or defined by the operator or the algorithm. That entails the problem that the so-called adhesive joint between the abutment and the prosthetic fitting is displaced in the direction of the dental implant and thus the jaw bone of the patient, which has destructive consequences for the gingiva and the jaw bone.

### SUMMARY OF THE INVENTION

The object of the invention is to provide a method of producing a three-dimensional computer model of a patient-individual abutment intended for a patient for a dental implant by means of a computer and a method of producing such an abutment for a dental implant, in which the computer model or the abutment produced in accordance therewith is better compatible for the gingiva and the jaw bone of the patient.

In addition the invention seeks to provide a computer program product including commands for carrying out the method, a computer-readable storage medium and a computer-readable data carrier including such commands as well as a data carrier signal which transmits such a computer program product and a computer for carrying out one of the methods according to the invention and/or for executing the computer program product according to the invention.

The steps of the methods according to the invention are carried out outside of a body of the patient as the invention starts from an already existing three-dimensional representation of a jaw of the patient at least in that region of the jaw where the abutment is to be placed.

According to a first variant of the invention, a plate-like configuration of the coronally looking platform surface is provided beneath the preparation limit for the three-dimensional geometry of the platform, as known per se from EP 2 825 124 B1. The plate-like configuration has an adhesive joint, which is advantageously far away from the dental implant, between the abutment and the prosthetic fitting. Since it is further provided that a material thickness of the platform of the abutment is selected by an operator of the computer starting from the defined emergence profile, i.e. beneath the preparation limit, the operator of the computer no longer has any need to select a preparation limit which in itself extends too deeply, in order to increase his room for maneuver, as the invention allows the operator in advance to design the three-dimensional geometry of the abutment beneath the preparation limit. In order to avoid configurations which are detrimental here and which in the case of an abutment produced in accordance with the three-dimensional computer model provided by the computer could lead to a failure of the abutment the invention provides that the three-dimensional geometry of the abutment can be selected below the preparation limit by the operator only within a predetermined region saved in an electronic memory.

Per se a plate-like configuration of the coronally looking platform surface would be solely sufficient. Preferably however, there is also an underside of the platform, that is of a curved configuration in the coronal direction (the surface of the platform displaced from the coronally looking platform surface by the thickness of material).

The three-dimensional geometry of the abutment beneath the preparation limit can be selected by the operator at least to the extent that the operator, starting from the defined emergence profile, can select a material thickness for the platform of the abutment (i.e. the normal distance between the coronally looking platform surface and the platform surface remote therefrom).

In preferred embodiments, the invention provides that the three-dimensional geometry of the abutment beneath the preparation limit can be further selected by the operator to the extent that an operator of the computer selects
a material thickness of a wall of the screw passage (i.e. the normal distance between the inner wall defining the screw passage and a wall remote from said inner wall of the coronally extending region of the abutment, that has the screw passage), and/or
a material thickness of the base of the abutment (i.e. the normal distance between the outside of the abutment, that faces towards the connecting structure, and the coronally looking platform surface), and/or
for the transition between the coronally looking platform surface and the base of the abutment and/or for the transition between the base of the abutment and a wall of the screw passage a transition radius within a predetermined range saved in an electronic memory.

It should be noted that constant material thicknesses are preferred for the material thickness of the platform of the abutment and/or for the material thickness of the wall of the screw passage and/or for the material thickness of the base of the abutment. Basically however, a gradient would also be possible for one or more of those material thicknesses (variable material thickness). The gradient can be defined by predetermining one or more points of support.

Preferably, the respective range for the possible choice of the material thickness or the transition radius is selected in dependence on a material to be used for the abutment (for example ceramic, titanium, plastic or hybrid materials) and/or in dependence on the defined three-dimensional geometry of the abutment above the preparation limit.

The predetermined range saved in an electronic memory (to which the computer must naturally have access during execution of the method, at least during the corresponding step) can be respectively established on the basis of empirical values and/or on the basis of simulations and/or measurement series. For example, it can be provided that the material thickness
of the base of the abutment can be selected in a range of about 0.05 mm to about 4 mm,
of the platform can be selected in a range of about 0.05 mm to about 2 mm, and
of the wall of the screw passage can be selected in a range of about 0.05 mm to about 3 mm.

Preferably, the method and a corresponding computer program which in the execution of the program by a computer cause it to carry out the method, to directly provide a three-dimensional computer model for an abutment with a coronally looking platform surface of plate-like configuration starting from a provided three-dimensional representation readable by the computer of a jaw of the patient.

Alternatively, however, in a second variant of the invention it can be provided that firstly a three-dimensional start-computer-model of a patient-individual abutment is produced with a conventional method and a corresponding computer program. This abutment model is of such a form that a region extending radially between the screw passage of the abutment and the emergence profile of the abutment is represented as filled with material. The computer then carries out a transformation in accordance with the method pursuant to the invention or with a corresponding computer program pursuant to the invention in order to obtain from the three-dimensional start computer model the three-dimensional computer model. This three-dimensional model is provided beneath the preparation limit for the three-dimensional geometry of the platform with a plate-like configuration of the coronally looking platform surface and has the material thickness of the platform of the abutment, that is selected by an operator of the computer based on the defined emergence profile. The same additional optional features as discussed in relation to the first variant of the invention can be provided.

Preferably, an additive production method (for example laser sintering) in which powder (for example of ceramic, titanium, plastic or hybrid materials) is layer-wise hardened by the action of energy is proposed for the production of an abutment according to the invention for a dental implant using a material-reduced three-dimensional computer model of the patient-individualized abutment, said model being produced in accordance with a method according to at least one of the preceding embodiments.

In comparison with conventional machining or cutting methods for the production of an abutment additive production methods are distinguished by a reduced method operating time. In addition a machining or cutting method frequently requires a complicated re-clamping operation. A machining or cutting post-treatment may be required even when using an additive production method.

In all embodiments the screw passage can extend parallel to or at an angle relative to a central notional axis of the connecting structure of the abutment.

### BRIEF DESCRIPTION OF THE DRAWINGS

Embodiments of the invention are described with reference to the Figures in which:

FIGS. 1a, b are schematic views of two variants of an arrangement for carrying out the method according to the invention,

FIG. 2 is a schematic view of a production method of an abutment using the result of the method shown in FIG. 1a or 1b,

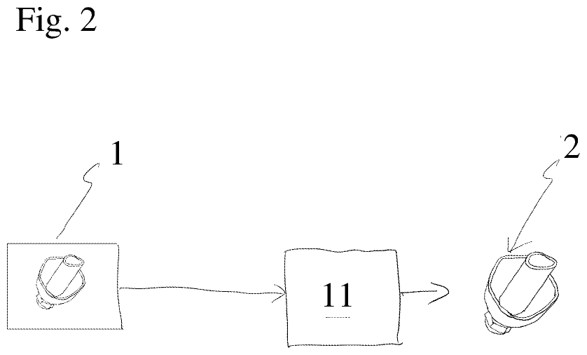

FIGS. 3a-d are various views and a sectional view of an embodiment of an abutment to be produced by the invention,

FIG. 4 is a sectional view as in FIG. 3d with dimensioning of the material thickness,

FIGS. 5a-d are various views and a sectional view of an embodiment of a three-dimensional start computer model of a patient-individual abutment which is transformed during the method according to the invention,

FIG. 6 is a sectional view through an embodiment of an abutment to be produced by the invention, wherein one can see how, in the second variant of the invention, the transformation is effected starting from a three-dimensional start computer model, and

FIGS. 7a-d are views of a computer-readable three-dimensional illustration of a jaw of a patient with a computer model of an abutment and without the abutment respectively, a gingiva mask and an abutment produced with the method according to the invention with a prosthetic fitting in the form of a crown.

### DETAILED DESCRIPTION OF THE INVENTION

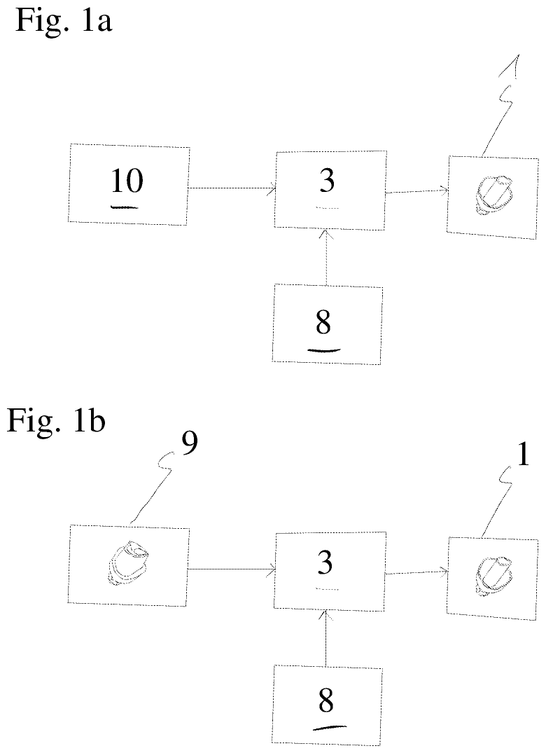

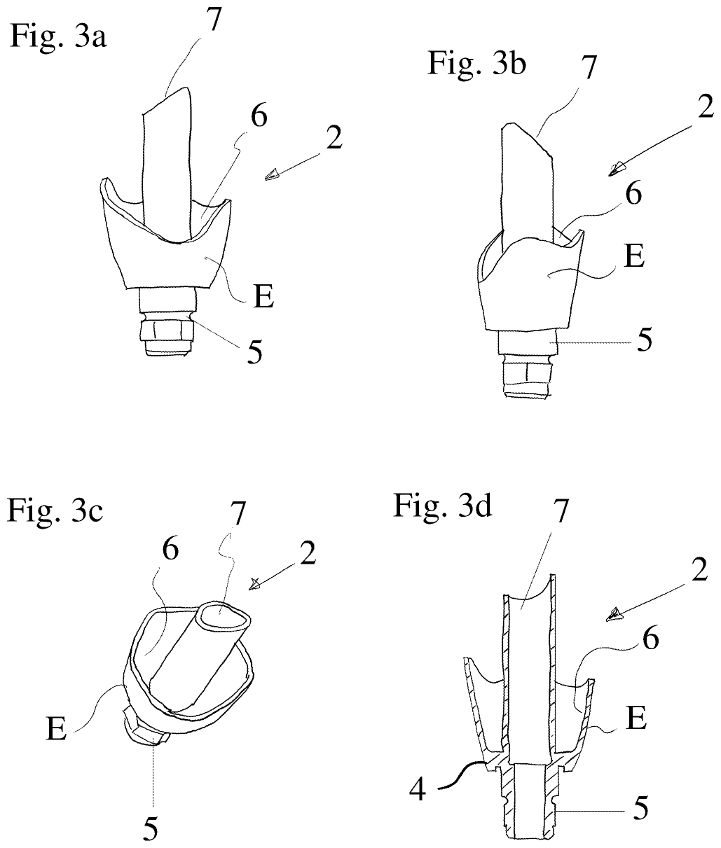

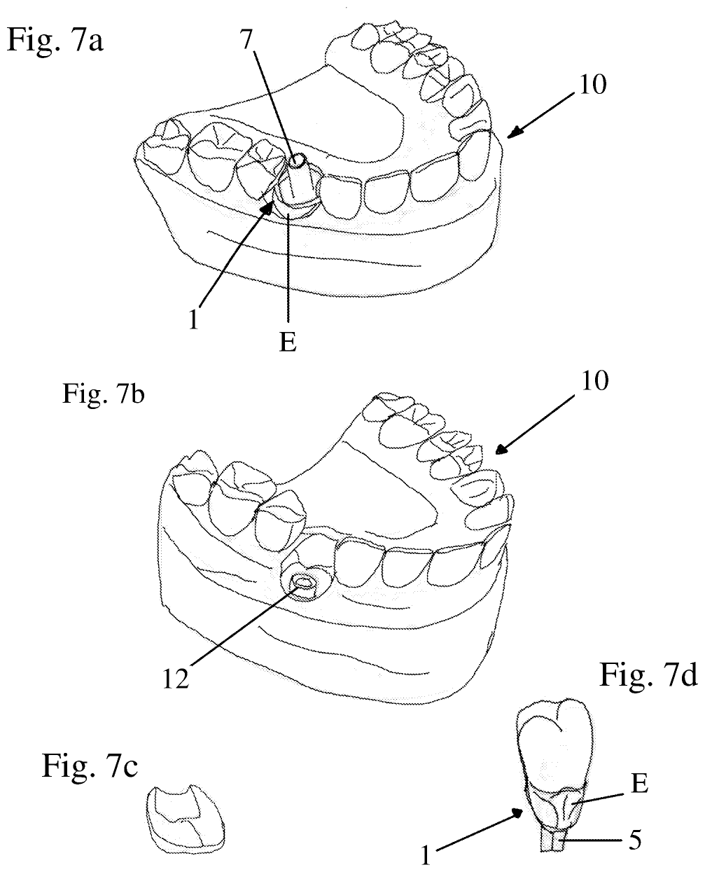

FIG. 1a schematically shows an arrangement for operating a method for the production of a three-dimensional computer model 1 of a patient-individual abutment 2 specific for a patient, for a dental implant 12 by means of a computer 3, wherein the abutment 2 (see FIGS. 3a-d) has:
a base 4,
a connecting structure 5 for connecting the abutment 2 to a pin-shaped dental implant 12 (see FIG. 7b),
a coronally looking platform surface 6 of a platform of the abutment 2 for supporting a prosthetic fitting, and
a screw passage 7 for fixing the abutment 2 to a pin-shaped dental implant 12.

A central notional axis Z of the connecting structure 5 of the abutment 2 can be seen here.

The procedure involves providing a three-dimensional representation 10, readable by the computer 3, of a jaw of the patient at least in that region of the jaw where the abutment 2 is to be placed. This three-dimensional representation can be produced in known manner either based on dental imprint of the jaw of the patient, that is digitized, or directly in digital form (for example using an intraoral scanner and/or a laboratory scanner).

In known manner selecting of a definition of a preparation limit in dependence on a gingiva configuration which is specific for the patient takes place where the abutment 2 is to be placed, by an operator of the computer 3 or by an algorithm.

A definition of a three-dimensional geometry of the abutment 2 above the preparation limit is implemented by an operator of the computer 3 or by an algorithm. The definition of the three-dimensional geometry of the abutment 2 can include:
Defining a position of the screw passage 7 extending in the abutment 2 for fixing the abutment 2 to a pin-shaped dental implant 12. Usually, the position is defined relative to an orientation of the pin-shaped dental implant 12.
Defining a width of a coronally looking platform surface 6 of the abutment 2.
Defining a height of the abutment 2.
Defining a width of a central raised portion on the abutment 2, that holds the screw passage.

A definition of the emergence profile E of the abutment is effected by an operator of the computer 3 or by an algorithm.

According to the invention, beneath the preparation limit for the three-dimensional geometry of the platform is a plate-like configuration of the coronally looking platform surface 6 provided.

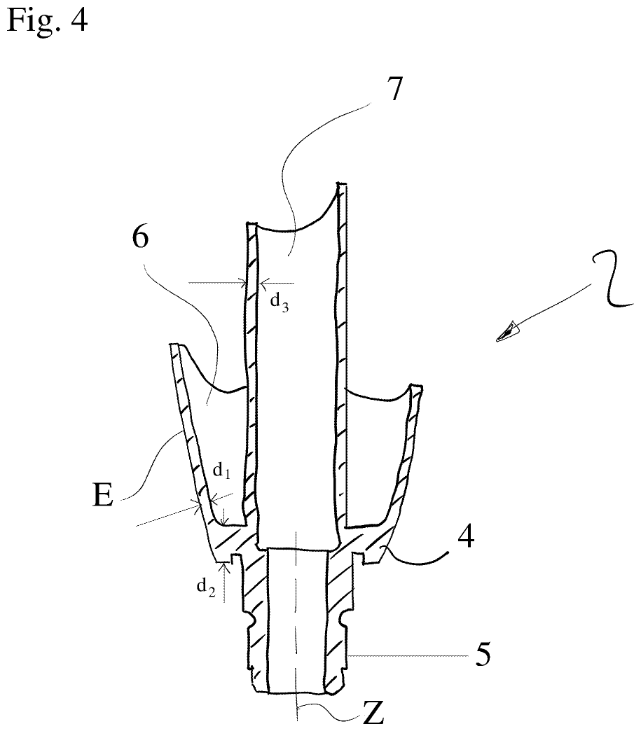

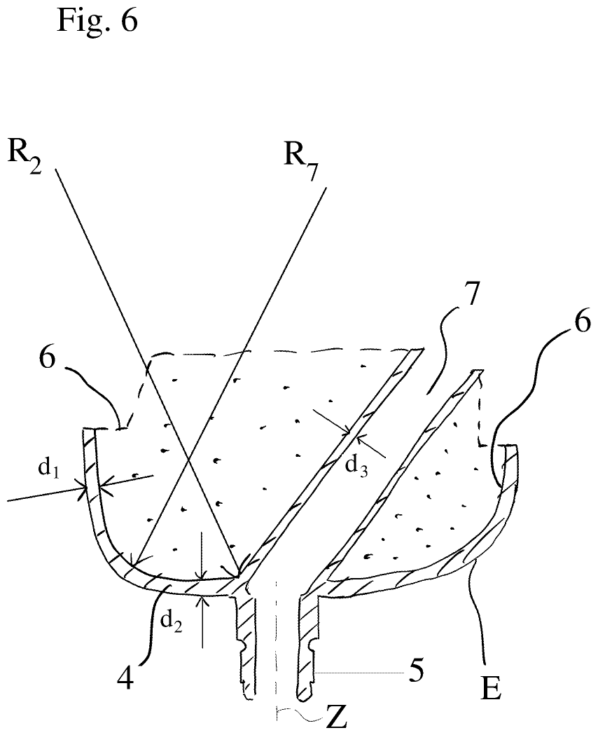

An operator of the computer 3, based on the defined emergence profile E, within predetermined regions saved in an electronic memory 8 (see FIG. 4 and FIG. 6) selects:
a material thickness d₁ of the platform of the abutment 2,
a material thickness d₂ of the base of the abutment 2 and/or
a material thickness d₃ of a wall of the screw passage 7 and/or
a transition radius R₁ for the transition between the coronally looking platform surface 6 and the base 4 of the abutment 2, and
a transition radius R₂ for the transition between the base of the abutment 2 and a wall of the screw passage 7.

The three-dimensional computer model 1 is provided by the computer 3, for example for the production of a patient-individual abutment 2 for a dental implant 12 using a three-dimensional computer model 1 produced in accordance with the above-described method, preferably by an additive production method.

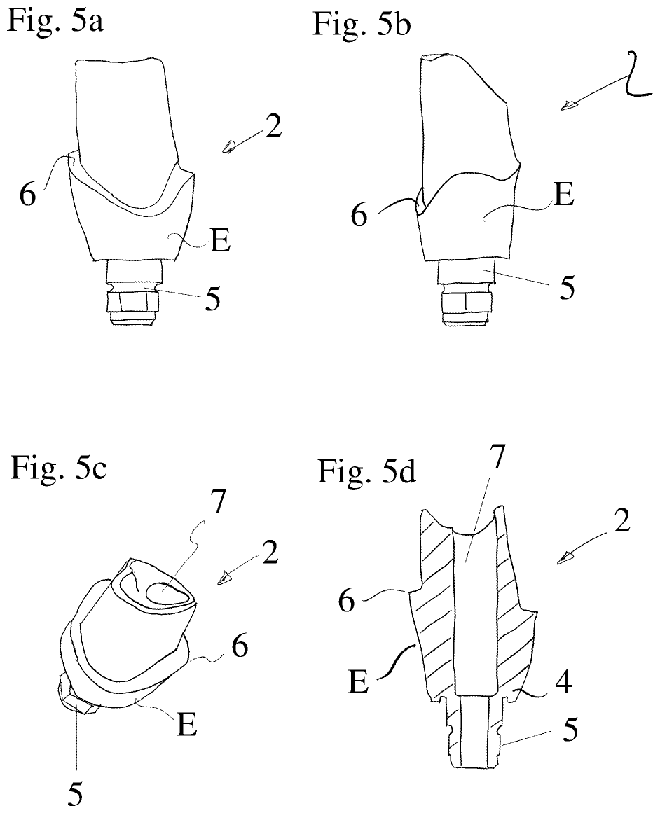

The embodiment of FIG. 1b differs from the previously discussed embodiment only in that, in accordance with the second variant of the invention, firstly a three-dimensional start computer model 9 of a patient-individual abutment 2 is produced in dependence on the provided three-dimensional representation of the jaw, the definition of the emergence profile E and the three-dimensional geometry of the abutment 2 above the preparation limit, wherein the three-dimensional start computer model 9 is of such a form that a region extending radially between the screw passage 10 of the abutment and the emergence profile E of the abutment 2 is represented as filled with material (see FIG. 5a-d). Starting from this three-dimensional start computer model 9 (which in itself represents a functional abutment) the computer 3 carries out a transformation by an algorithm (the algorithm for example computes a certain necessary material thickness depending on the computed loading peaks depending on the respective angle of the screw passage) in order to obtain from the three-dimensional start computer model 9 the three-dimensional computer model 1 in respect of which there is a plate-like configuration of the coronally looking platform surface 6 beneath the preparation limit for the three-dimensional geometry of the platform and which has the material thickness d₁ of the platform of the abutment 2 and the further discussed parameters, said material thickness being selected by an operator of the computer 3 based on the defined emergence profile E. FIG. 6 exemplifies what region (dotted region) that is shown as filled with material is to be removed in the transformation operation.

FIG. 7a shows a view of a three-dimensional representation 10 readable by the computer 3 of a jaw of a patient with a three-dimensional computer model 1 of a patient-individual abutment 2.

In FIG. 7b the abutment 2 shown in FIG. 7a has been removed, which allows a view of the upper region of a pin-shaped dental implant 12. The abutment 2 is inserted with its connecting structure 5 into that upper region of the pin-shaped dental implant 12. FIG. 7c shows a gingiva mask (which however is not provided in FIGS. 7a and 7b).

FIG. 7d shows an abutment 2 produced in accordance with the method of the invention, with a prosthetic fitting in the form of a crown.

### LIST OF REFERENCES

1 three-dimensional computer model of a patient-individual abutment
2 abutment
3 computer
4 base of the abutment
5 connecting structure
6 coronally looking platform surface
7 screw passage
8 electronic memory
9 three-dimensional start computer model of a patient-individual abutment
10 three-dimensional representation of a jaw of the patient
11 production installation
12 dental implant
E emergence profile of the abutment
Z central notional axis of the connecting structure of the abutment
d₁ material thickness of the platform of the abutment
d₂ material thickness of the base of the abutment
d₃ material thickness of a wall of the screw passage of the abutment
R₁ transition radius of a transition between the coronally looking platform surface and the base of the abutment
R₂ transition radius of a transition between the base of the abutment and a wall of the screw passage

1. A method of producing a three-dimensional computer model of a patient-individual abutment specific for a patient for a dental implant by means of a computer, wherein the abutment has at least a base, a connecting structure for connecting the abutment to a pin-shaped dental implant, a coronally looking platform surface of a platform of the abutment for supporting a prosthetic fitting and a screw passage for fixing the abutment to a pin-shaped dental implant, including at least the following steps:
providing a three-dimensional representation readable by the computer of a jaw of the patient at least in that region of the jaw, where the abutment is to be placed,
selecting a definition of a preparation limit in dependence on a gingiva configuration which is individual for the patient, where the abutment is to be placed, by an operator of the computer or by an algorithm,
defining a three-dimensional geometry of the abutment above the preparation limit by an operator of the computer or by an algorithm,
defining an emergence profile of the abutment by an operator of the computer or by an algorithm, and
providing the three-dimensional computer model by the computer,
wherein below the preparation limit for the three-dimensional geometry of the platform is a plate-like configuration of the coronally looking platform surface provided and a material thickness of the platform of the abutment is selected by an operator of the computer starting from the defined emergence profile within a predetermined range saved in an electronic memory.

2. A method of producing a three-dimensional computer model of a patient-individual abutment specific for a patient for a dental implant by means of a computer, wherein the three-dimensional start computer model represents an abutment which has a base, a connecting structure for connecting the abutment to a pin-shaped dental implant, a coronally looking platform surface of a platform of the abutment for supporting a prosthetic fitting and a screw passage for fixing the abutment to a pin-shaped dental implant, comprising at least the following steps:
providing a three-dimensional start computer model of the patient-individual abutment, wherein the three-dimensional start computer model is of such a form that a region extending radially between the screw passage of the abutment and the emergence profile of the abutment is represented as filled with material,
carrying out a transformation by means of the computer in order to obtain from the three-dimensional start computer model a three-dimensional computer model in which below the preparation limit for the three-dimensional geometry of the platform is a plate-like configuration of the coronally looking platform surface provided and which has a material thickness of the platform of the abutment, that is selected by an operator of the computer starting from the defined emergence profile within a predetermined range saved in an electronic memory.

3. The method as set forth in claim 1, wherein further a material thickness of a wall of the screw passage is selected within a predetermined region saved in an electronic memory by an operator of the computer.

4. The method as set forth in claim 1, wherein a material thickness of the base of the abutment is selected within a predetermined range saved in an electronic memory by an operator of the computer.

5. The method as set forth in claim 1, wherein a transition radius within a predetermined region saved in an electronic memory is selected:
for the transition between the coronally looking platform surface and the base of the abutment, and/or
for the transition between the base of the abutment and a wall of the screw passage.

6. The method as set forth in claim 1, wherein the respective range for the possible selection of the material thickness and/or for the transition radius respectively is selected in dependence on a material to be used for the abutment and/or in dependence on the defined three-dimensional geometry of the abutment above the preparation limit.

7. A method of producing a patient-individual abutment for a dental implant using a three-dimensional computer model produced in accordance with the method as set forth in claim 1, preferably by an additive production method.

8. A computer program product including commands which when the program is executed by a computer cause it to carry out the method as set forth in claim 1.

9. A computer-readable storage medium including commands which when the program is executed by a computer cause it to carry out the method as set forth in claim 1.

10. A computer-readable data carrier on which the computer program product as set forth in claim 8 is saved.

11. A data carrier signal which transmits the computer program product as set forth in claim 8.

12. A computer configured to execute the computer program product as set forth in claim 8.
